(19) United States
(12) Patent Application Publication
(10) Pub. No.: US 2020/0383865 A1
(43) Pub. Date: Dec. 10, 2020

(54) PADDED AND PREWIRED EXOSKELETON HARNESS

(71) Applicant: Lockheed Martin Corporation, Bethesda, MD (US)

(72) Inventors: Gavin A. Barnes, St. Cloud, FL (US); Sean A. Nelson, Orlando, FL (US); Christine Sleppy, Orlando, FL (US)

(21) Appl. No.: 16/970,892

(22) PCT Filed: Feb. 25, 2019
(86) PCT No.: PCT/US19/19431

§ 371 Date: Aug. 18, 2020

Related U.S. Application Data
(60) Provisional application No. 62/635,167, filed on Feb. 26, 2018.

(51) Int. Cl. A61H 3/00; A41D 1/06; A41D 1/00; A61B 5/00; A41D 13/00; A61B 5/0488; A41D 13/05

(52) U.S. Cl. CPC A61H 3/00; A41D 1/06; A41D 1/005; A61B 5/6804; A41D 13/0007; A61B 5/0488; A61B 2562/223; A61H 2003/007; A61H 2201/1652; A41D 2400/32; A61H 2201/5097; A61B 2562/222; A41D 13/0543

(57) ABSTRACT

Disclosed herein is a padded and prewired exoskeleton harness. The exoskeleton harness comprises a clothing article including a clothing material to receive at least a portion of a user. In certain embodiments, the clothing material includes a fabric and at least one patch with a greater coefficient of static friction and greater thickness than the fabric to increase the friction between the user and the exoskeleton, thereby increasing coupling and power transfer therebetween. In certain embodiments, the exoskeleton harness includes at least one internal electronic port positioned at an interior of the clothing material to electronically communicate with one of a plurality of skin-mounted biosensors. The internal ports are prewired within the clothing material to an external electronic a port for communicating with the exoskeleton. The prewiring of the exoskeleton harness facilitates connection between skin-mounted sensors and a data acquisition (DAQ) system.

### RELATED APPLICATION

This application claims the benefit of U.S. Provisional Application No. 62/635,167, filed on Feb. 26, 2018, entitled “Padded and Prewired Exoskeleton Harness,” the disclosure of which is hereby incorporated herein by reference in its entirety.

### TECHNICAL FIELD

The embodiments relate to exoskeleton harnesses, and in particular, to a padded and prewired exoskeleton harness for an exoskeleton.

### BACKGROUND

An exoskeleton is a device external to a user, which provides support and/or protection (e.g., increased strength). Users typically wear a layer of clothing material between the user and the exoskeleton, such as pants (e.g., jeans, fatigues, etc.). However, such clothing material may complicate mounting of the exoskeleton on a user, and/or may negatively impact performance. For example, in use, loose clothing can be a hindrance to harnessing, especially for conformal exoskeletons, as the loose clothing may bunch up, thereby creating abrasive hotspots. Further, such loose clothing may increase the chances of the exoskeleton slipping on the user, increasing abrasion and discomfort.

Exoskeletons may use skin-mounted biosensors to provide biofeedback to the exoskeleton, thereby allowing the exoskeleton to follow the motion of the user. Mounting such biosensors and exoskeletons can be time consuming and complicated as the wires from the biosensors must be routed up and around the pants, leading to potential disorganization and discomfort. Further, if a user gets out of the exoskeleton while instrumented, the user may be covered with a collection of hanging wires, further encumbering the user.

### SUMMARY

Disclosed herein is a padded and prewired exoskeleton harness. The exoskeleton harness comprises one or more clothing articles (e.g., shirt, pants, etc.). The clothing article of the exoskeleton harness (e.g., each of the shirt and pants) includes a clothing material to receive at least a portion of a user (e.g., upper torso, arms, lower torso, legs, etc.). In certain embodiments, the clothing material includes a fabric and at least one patch (i.e., padding) at an exterior surface of the clothing material. The patch has a greater coefficient of static friction and greater thickness than the fabric. The exoskeleton harness is configured to align with and contact coupling portions of the exoskeleton. In this way, the exoskeleton harness increases the friction between the user and the exoskeleton, thereby increasing coupling and power transfer therebetween. In certain embodiments, the exoskeleton harness includes at least one internal electronic port positioned at an interior of the clothing material to electronically communicate with one of a plurality of skin-mounted biosensors. The internal ports are prewired within the clothing material to an external electronic port for communicating with the exoskeleton. In this way, the prewiring of the exoskeleton harness facilitates connection between skin-mounted sensors and a data acquisition (DAQ) system, such as for use in additional processes by the exoskeleton (e.g., actuation control, user health monitoring, etc.).

In one embodiment, the exoskeleton harness includes a clothing material defining an interior cavity. The clothing material is configured to receive at least a portion of a user within the interior cavity. The clothing material includes a fabric and at least one patch. Only a portion of an exterior surface of the clothing material comprises the at least one patch. The at least one patch has a greater coefficient of static friction and greater thickness than the fabric. The exoskeleton harness is configured such that the at least one patch is positioned to align with and contact at least one coupling portion of an exoskeleton.

In another embodiment, the exoskeleton harness includes a clothing material, an external electronic port, at least one internal electronic port, and at least one transmission path. The clothing material defines an interior cavity. The clothing material is configured to receive at least a portion of a user within the interior cavity. The clothing material has a thickness extending between an exterior and an interior of the clothing material. The external electronic port is positioned at the exterior of the clothing material and is configured to electronically communicate with a first electronic device separate from the exoskeleton harness. The at least one internal electronic port is positioned at the interior of the clothing material within the interior cavity. The at least one internal electronic port is configured to electronically communicate with at least one second electronic device separate from the exoskeleton harness. The at least one transmission path is embedded within the clothing material and communicatively connects the external port to the at least one internal port.

In another embodiment, the exoskeleton harness includes a clothing material, an external electronic port, at least one internal electronic port, and at least one transmission path. The clothing material defines an interior cavity. The clothing material is configured to receive at least a portion of a user within the interior cavity. The clothing material has a thickness extending between an exterior and an interior of the clothing material. The clothing material includes a fabric and at least one patch. Only a portion of an exterior surface of the clothing material comprises the at least one patch. The at least one patch has a greater coefficient of static friction and greater thickness than the fabric. The external electronic port is positioned at the exterior of the clothing material and is configured to electronically communicate with an exoskeleton. The at least one internal electronic port is positioned at the interior of the clothing material within the interior cavity. The at least one internal electronic port is configured to electronically communicate with one of a plurality of skin-mounted biosensors, such as an electromyography biosensor. The at least one transmission path is embedded within the clothing material and communicatively connects the external port to the at least one internal port. The exoskeleton harness is configured such that the at least one patch is positioned to align with and contact at least one coupling portion of the exoskeleton.

Those skilled in the art will appreciate the scope of the disclosure and realize additional aspects thereof after reading the following detailed description of the embodiments in association with the accompanying drawing figures.

### BRIEF DESCRIPTION OF THE DRAWINGS

The accompanying drawing figures incorporated in and forming a part of this specification illustrate several aspects of the disclosure, and together with the description serve to explain the principles of the disclosure.

FIG. 1 is a partial cross-sectional front view of an exoskeleton harness worn by a user with part of an exoskeleton coupled to one side of the exoskeleton harness;

FIG. 2 is a back view of the exoskeleton harness of FIG. 1 worn by a user, the exoskeleton harness including a shirt and pants;

FIG. 3 is a partial cross-sectional view of the exoskeleton harness of FIGS. 1 and 2 illustrating internal ports prewired with an external port;

FIG. 4 is a cross-sectional side view of an internal prewired port of FIGS. 1 and 3 electronically connected to a skin-mounted biosensor attached to skin of a user;

FIG. 5A is a cross-sectional perspective view of a clothing material of the exoskeleton harness of FIGS. 1-3 illustrating a transmission path embedded within a single layer of the clothing material;

FIG. 5B is a cross-sectional perspective view of a clothing material of the exoskeleton harness of FIGS. 1-3 illustrating a transmission path embedded within two layers of the clothing material;

FIG. 6 is a front view of the exoskeleton harness of FIGS. 1-3 illustrating the plurality of patches which provide padding between the user and an exoskeleton;

FIG. 7A is a cross-sectional side view of the clothing material of the exoskeleton harness of FIGS. 1-3 and 6 illustrating a fabric and a single layer patch;

FIG. 7B is a cross-sectional side view of the clothing material of the exoskeleton harness of FIGS. 1-3 and 6 illustrating a fabric and a multilayer patch;

FIG. 7C is a top view of the clothing material of the exoskeleton harness of FIGS. 1-3 and 6 illustrating a patch with a checkered pattern of a gripping layer;

FIG. 8A is a cross-sectional view of an annular patch of the exoskeleton harness of FIGS. 1-3 and 6 illustrating a uniform annular thickness;

FIG. 8B is a cross-sectional view of an annular patch of the exoskeleton harness of FIGS. 1-3 and 6 illustrating a variable annular thickness;

FIG. 9A is a front view of the pants of the exoskeleton harness of FIGS. 1-3 and 6;

FIG. 9B is a side view of the pants of FIG. 9A;

FIG. 9C is a front perspective view of the pants of the exoskeleton harness of FIGS. 9A and 9B with an exoskeleton mounted to the pants of the exoskeleton harness; and

FIG. 9D is a side view of the pants of the exoskeleton harness of FIGS. 9A-9C with the exoskeleton mounted to the pants of the exoskeleton harness.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

The use herein of ordinals in conjunction with an element is solely for distinguishing what might otherwise be similar or identical labels, such as “first format” and “second format,” and does not imply a priority, a type, an importance, or other attribute, unless otherwise stated herein. The term “about” used herein in conjunction with a numeric value means any value that is within a range of ten percent greater than or ten percent less than the numeric value.

The use herein of “annular” means surrounding, which may include circular shapes or any other type of shape.

The use herein of “proximate” means at, next to, or near.

As used herein “clothing,” “clothes,” and/or “clothing articles” means an item made of a clothing material which is worn to cover at least a portion of a body including, for example, shirts, pants, etc. As used herein “shirt” means a garment for an upper body of a person made of a clothing material. As used herein “pants” means a garment for a lower body of a person made of a clothing material. As used herein “clothing material” means the material (e.g., fabric) which defines the clothing article. As used herein “fabric” means a flexible material made of fibers (e.g., natural or synthetic fibers).

Disclosed herein is a padded and prewired exoskeleton harness. The exoskeleton harness comprises one or more clothing articles (e.g., shirt, pants, etc.). The clothing article of the exoskeleton harness (e.g., each of the shirt and pants) includes a clothing material to receive at least a portion of a user (e.g., upper torso, arms, lower torso, legs, etc.). In certain embodiments, the clothing material includes a fabric and at least one patch (i.e., padding) covering at least a portion of the exterior surface of the clothing material. The patch has a greater coefficient of static friction and greater thickness than the fabric. The exoskeleton harness is configured to align with and contact coupling portions of the exoskeleton. In this way, the exoskeleton harness increases the friction between the user and the exoskeleton, thereby increasing coupling and power transfer therebetween. In certain embodiments, the exoskeleton harness includes at least one internal electronic port positioned at an interior of the clothing material to electronically communicate with one of a plurality of skin-mounted biosensors. The internal ports are prewired within the clothing material to an external electronic port for communicating with the exoskeleton. In this way, the prewiring of the exoskeleton harness facilitates connection between skin-mounted sensors and a data acquisition (DAQ) system, such as for use in additional processes by the exoskeleton (e.g., actuation control, user health monitoring, etc.).

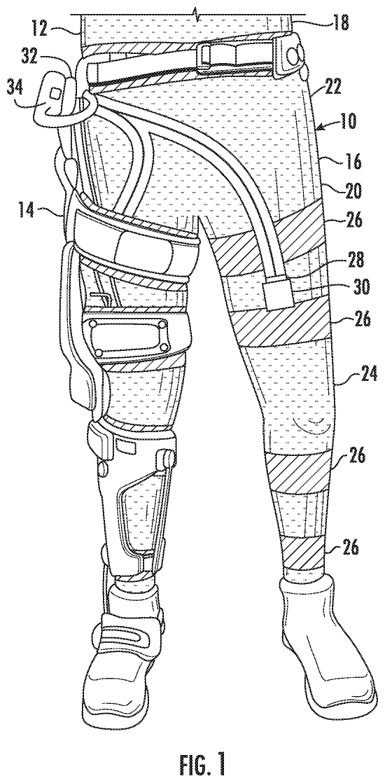

FIG. 1 is a partial cross-sectional front view of an exoskeleton harness 10 worn by a user 12 with part of an exoskeleton 14 coupled to one side of the exoskeleton harness 10. In particular, the exoskeleton harness 10 is padded and prewired. The exoskeleton harness 10 includes one or more clothing articles 16 (e.g., shirt 18, pants 20, etc.). The clothing article 16 of the exoskeleton harness 10 (e.g., each of the shirt 18 and pants 20) includes a clothing material 22 to receive at least a portion of a user 12 (e.g., upper torso, arms, lower torso, legs, etc.). In certain embodiments, the clothing material 22 includes a fabric 24 and at least one patch 26 (i.e., padding) at an exterior surface of the clothing material 22. The patch 26 has a greater coefficient of static friction and greater thickness than the fabric 24. The exoskeleton harness 10 is configured to align with and contact coupling portions of the exoskeleton 14. In this way, the exoskeleton harness 10 increases the friction between the user 12 and the exoskeleton 14, thereby increasing coupling and power transfer therebetween. In certain embodiments, the exoskeleton harness 10 includes at least one internal electronic port 28 positioned at an interior of the clothing material 22 to electronically communicate with one of a plurality of skin-mounted biosensors 30. The internal ports 28 are prewired within the clothing material 22 to an external electronic port 32 for communicating with the exoskeleton 14. In this way, the prewiring of the exoskeleton harness 10 facilitates connection between skin-mounted biosensors 30 and a data acquisition (DAQ) system 34, such as for use in additional processes by the exoskeleton 14 (e.g., actuation control, user health monitoring, etc.).

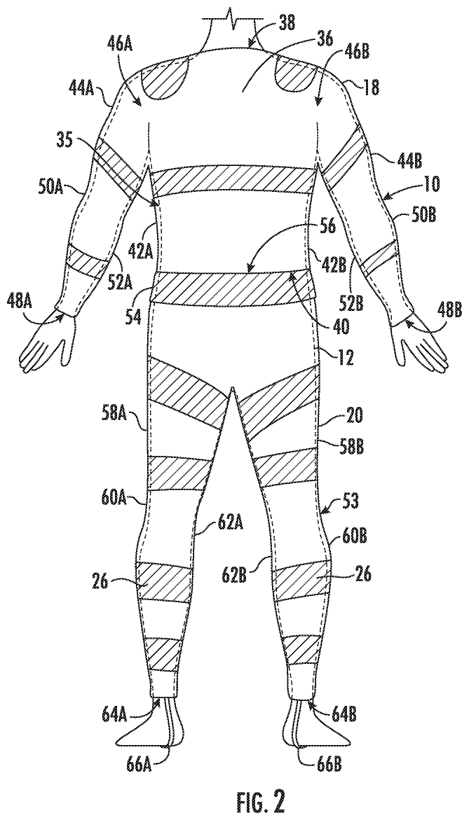

FIG. 2 is a back view of the exoskeleton harness 10 of FIG. 1 worn by a user 12. The exoskeleton harness 10 includes a shirt 18 and pants 20. The shirt 18 and the pants 20 are two separate clothing articles. However, in certain embodiments, the shirt 18 and the pants 20 are integrally connected (e.g., like a wetsuit).

The shirt 18 is illustrated as a long sleeved shirt. In certain embodiments, the shirt 18 may be a short sleeved shirt (e.g., T-shirt), sleeveless shirt, etc. The shirt 18 is configured to receive at least a portion of the user within a shirt interior cavity 35 defined by the clothing material 22 of the shirt 18. In other words, the clothing material 22 of the shirt 18 defines a shirt interior cavity 35 and is configured to receive at least a portion of the user 12 within the shirt interior cavity 35.

The shirt 18 includes a body portion 36 configured to receive a body (e.g., chest, torso, etc.) of the user 12. The body portion 36 includes a neck hole 38 configured to receive at least a portion of a neck of the user 12 and a waist hole 40 configured to receive at least a portion of a waist of the user 12. Further, the body portion 36 includes a left side seam 42A and a right side seam 42B. The shirt 18 includes a left sleeve 44A to receive at least a portion of a left arm (e.g., upper arm, lower arm, wrist, etc.). The left sleeve 44A includes a left arm hole 46A (proximate the body portion 36) and a left sleeve opening 48A (opposite the body portion 36). The left sleeve 44A further includes a left top seam 50A and a left bottom seam 52A. The shirt 18 further includes a right sleeve 44B to receive at least a portion of a right arm (e.g., upper arm, lower arm, wrist, etc.). The right sleeve 44B includes a right arm hole 46B (proximate the body portion 36) and a right sleeve opening 48B (opposite the body portion 36). The right sleeve 44B further includes a right top seam 50B and a right bottom seam 52B.

The pants 20 are illustrated as long pants. In certain embodiments, the pants 20 may be shorts, etc. The pants 20 are configured to receive at least a portion of the user within a pants interior cavity 53 defined by the clothing material 22 of the pants 20. In other words, the clothing material 22 of the pants 20 defines a pants interior cavity 53 and is configured to receive at least a portion of the user 12 within the pants interior cavity 53.

The pants 20 include a waist band 54 defining a waist opening 56. The pants 20 further include a left pant leg 58A to receive at least a portion of a left leg (e.g., upper leg, lower leg, ankle, etc.) of the user 12. The left pant leg 58A includes a left side seam 60A and a left inseam 62A. The left pant leg 58A includes a left leg opening 64A (opposite the waistband 54) to receive at least a portion of the left leg (e.g., ankle) of a user 12 therethrough. In certain embodiments, the left pant leg 58A further includes a left stirrup 66A (proximate the left leg opening 64A) to receive at least a portion of a left foot of a user 12. The pants 20 further include a right pant leg 58B to receive at least a portion of a right leg (e.g., upper leg, lower leg, ankle, etc.) of the user 12. The right pant leg 58B includes a right side seam 60B and a right inseam 62B. The right pant leg 58B includes a right leg opening 64B (opposite the waistband 54) to receive at least a portion of the right leg (e.g., ankle) of a user 12 therethrough. In certain embodiments, the right pant leg 58B further includes a right stirrup 66B (proximate the right leg opening 64B) to receive at least a portion of a right foot of a user 12. The left stirrup 66A and right stirrup 66B prevent the pants 20 from shifting upward on a user 12 to ensure that the patches 26 are properly positioned relative to the user 12. For example, during use, the bottom of the pants 20 may shift upward exposing skin of the user 12 to the environment and the exoskeleton 14. Additionally or alternatively, in certain embodiments, the pants 20 include elastic around the left leg opening 64A and/or the right leg opening 64B to prevent the pants from shifting upward on a user 12.

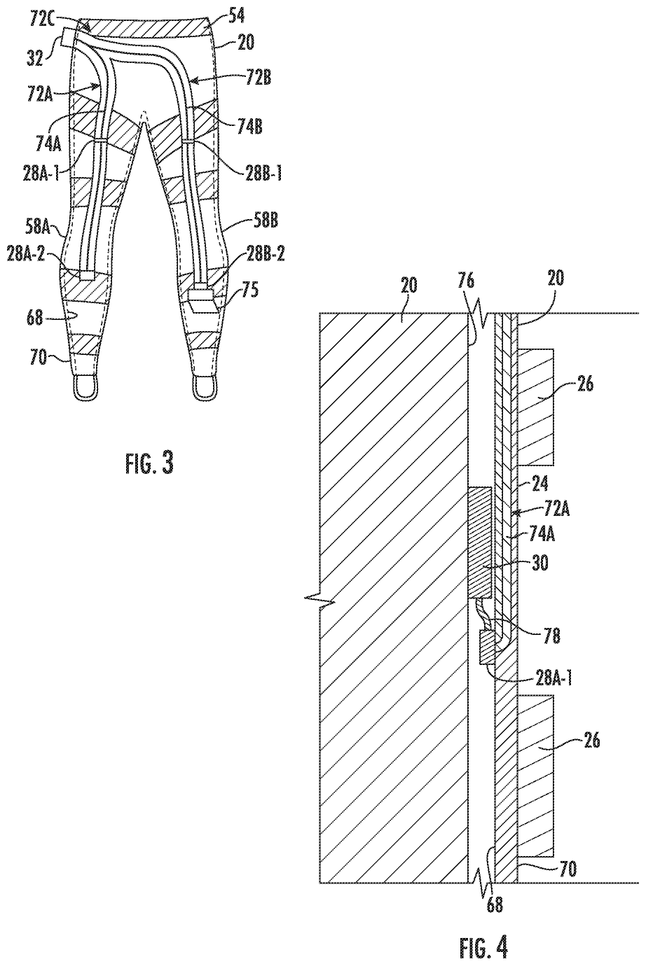

FIGS. 3-9D discuss the pants 20 of the exoskeleton harness 10 in more detail. However, it is noted that the features discussed with respect to the pants 20 are also applicable to the shirt 18 and/or other clothing articles.

FIG. 3 is a partial cross-sectional view of the exoskeleton harness 10 of FIGS. 1 and 2 illustrating internal ports 28 prewired with an external port 32. In particular, the internal ports 28 are positioned at an interior 68 (e.g., interior surface) of the pants 20 and the external port 32 is positioned at an exterior 70 of the pants 20. The internal ports 28 are configured to electronically and/or mechanically connect to skin-mounted biosensors 30 (see FIG. 1), which provide biofeedback to the exoskeleton. Prewiring of the pants 20 of the exoskeleton harness 10 facilitates ease of use of the exoskeleton harness 10 and exoskeleton 14 (see FIG. 1), particularly in that the user 12 does not have to run wires from the skin-mounted biosensor 30 (see FIG. 1) (e.g., electromyography (EMG) sensor) up the pants 20 (e.g., between the pants 20 and the user 12).

The internal ports 28 include a left upper internal port 28A-1, a left lower internal port 28A-2, a right upper internal port 28B-1, and a right lower internal port 28B-2. In certain embodiments, the internal ports 28 include fewer or more internal ports 28. The left upper internal port 28A-1 is positioned at an interior 68 and in an upper left leg portion of the pants 20 to be positioned proximate an upper left leg of the user 12 (e.g., for communication with a biosensor attached to the upper left leg of the user 12). The left lower internal port 28A-2 is positioned at an interior 68 and in a lower left leg portion of the pants 20 to be positioned proximate a lower left leg of the user 12 (e.g., for communication with a biosensor attached to the lower left leg of the user 12). The right upper internal port 28B-1 is positioned at an interior 68 and in an upper right leg portion of the pants 20 to be positioned proximate an upper right leg of the user 12 (e.g., for communication with a biosensor attached to the upper right leg of the user 12). The right lower internal port 28B-2 is positioned at an interior 68 and in a lower right leg portion of the pants 20 to be positioned proximate a lower right leg of the user 12 (e.g., for communication with a biosensor attached to the lower right leg of the user 12).

The pants 20 of the exoskeleton harness 10 define channels 72 within a thickness of the clothing material 22 of the pants 20. The thickness of the clothing material 22 extends between an exterior and an interior of the clothing material 22. In certain embodiments, the thickness of the clothing material is between 6 mm and 75 mm, between 12 mm and 50 mm, between 25 mm and 50 mm, between 25 mm and 37 mm, etc. The channels 72 include a left channel 72A along the length of the left pant leg 58A and a right channel 72B along the length of the right pant leg 58B. The left channel 72A and right channel 72B branch from a common channel 72C at an upper part of the pants 20 (proximate the waistband 54 of the pants 20).

The left channel 72A extends down the left pant leg 58A from the external port 32 to one or more of the left internal ports 28A-1, 28A-2. A left transmission path 74A (e.g., wire, optical fiber, cable with multiple wires, cable with multiple fibers, etc.) is positioned in the left channel 72A within a thickness of the clothing material 22 of the pants 20, communicatively connecting (e.g., optically, electrically, etc.) the left internal ports 28A-1, 28A-2 with the external port 32. In other words, one end of the transmission path 74A terminates at the interior 68 of the exoskeleton harness 10, and another end of the transmission path 74A terminates at an exterior 70 of the exoskeleton harness 10. The left upper internal port 28A-1 may be connected in series or in parallel with the left lower internal port 28A-2.

Similarly, the right channel 72B extends down the right pant leg 58B from the external port 32 to one or more of the right internal ports 28B-1, 28B-2. A right transmission path 74B (e.g., wire, optical fiber, cable with multiple wires, cable with multiple fibers, etc.) is positioned in the right channel 72B within a thickness of the clothing material 22 of the pants 20, communicatively connecting (e.g., optically, electrically, etc.) the right internal ports 28B-1, 28B-2 with the external port 32. In other words, one end of the transmission path 74B terminates at the interior 68 of the exoskeleton harness 10, and another end of the transmission path 74B terminates at an exterior 70 of the exoskeleton harness 10. The right upper internal port 28B-1 may be connected in series or in parallel with the right lower internal port 28B-2.

In certain embodiments, the exoskeleton harness 10 includes one or more access panels 75 (e.g., flaps) that provide access to the user's skin at various locations on the user's body to facilitate attachment or detachment of the biosensors, while the user is wearing the exoskeleton harness 10. In this way, the user could first put on the pants 20, and then connect the internal ports 28A, 28B with the biosensors.

The external port 32 (i.e., plug) is positioned at the exterior 70 of the pants 20, proximate the waistband 54 at an upper portion of the pants. This provides ease of use for a user to operate the external port 32 and/or connect the external port 32 to the exoskeleton 14. The external port 32 may comprise one or more ports in a consolidated location on the pants 20 to easily mechanically and/or electronically connect with the exoskeleton 14 and/or onboard data acquisition (DAQ) system 34 (see FIG. 1). In certain embodiments, the external port 32 is coupled to the electronics associated with a force-amplified exoskeleton 14. In certain embodiments, the external port 32 provides a central location to mechanically and electronically connect to the exoskeleton 14 or other electronic device. Additionally or alternatively, in certain embodiments, the external port 32 provides a central location for wirelessly communicating with the exoskeleton or other electronic device.

FIG. 4 is a cross-sectional side view of an internal prewired port 28 of FIGS. 1 and 3 electronically connected to a skin-mounted biosensor 30 (e.g., epidermal electronics-based sensors) attached to skin 76 of a user 12. The skin-mounted biosensors 30 may be attached to the user 12 in a variety of ways including adhered and/or strapped, etc. In certain embodiments, the skin-mounted biosensors 30 measure heart rate monitoring, body temperature, etc.

As noted above, the internal port 28A-1 is positioned at an interior 68 of the pants 20, and the transmission path 74A is embedded within a thickness of a clothing material 22 of the pants 20 (where the thickness is defined between an interior 68 and exterior 70 of the clothing material 22 of the pants 20). In particular, the transmission path 74A is embedded within a thickness of the fabric 24 of the clothing material 22. Accordingly, as a user 12 puts on the pants, and rather than run wires within the pants interior cavity 53 of the pants 20, the user 12 may simply connect the skin-mounted biosensors 30 to the internal port 28. In particular, a user 12 can mount the skin-mounted biosensors 30, and then, as the user 12 pulls the pants up, the user 12 can simply plug the small lead 78 of the skin-mounted biosensor 30 into the internal port 28A-1. The connection between the internal port 28A-1 and the skin-mounted biosensor 30 could include a nano miniature polarized PZN connector, micro USB connector, Z-ray connector, etc.

In certain embodiments, the internal port 28A-1 is wirelessly connected to the skin-mounted biosensors 30 (e.g., partially by proximity to the skin-mounted biosensors 30). For example, the skin-mounted biosensors 30 may include near-field communication (NFC) (e.g., NFC based epidermal electronics), such as those provided by Stretch Med. In some of these embodiments, the internal port 28A-1 is positioned proximate the respective skin-mounted biosensor 30 for wireless communication therewith.

Once the pants 20 are pulled up, a user 12 can then easily connect the pants 20 to the exoskeleton 14 at the external port 32. Such a configuration decreases time, effort, and complexity in a user 12 coupling to an exoskeleton 14.

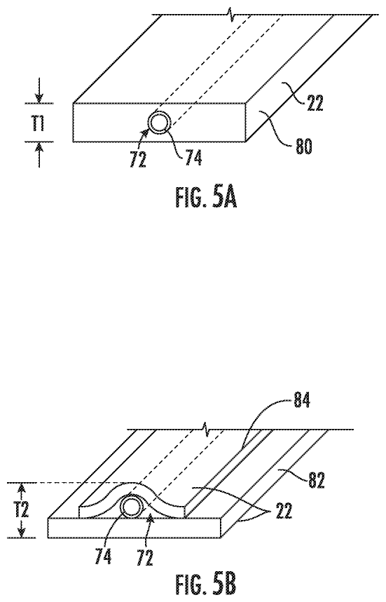

FIGS. 5A and 5B illustrate embodiments for embedding the transmission path 74 within a thickness of the clothing material 22 of the pants 20. In particular, FIG. 5A is a cross-sectional perspective view of a clothing material 22 of the exoskeleton harness 10 of FIGS. 1-3 illustrating a transmission path 74 embedded within a thickness T1 of a single layer 80 of the clothing material 22. The channel 72 is formed within the single layer 80 of the clothing material 22. Accordingly, the clothing material 22 has a thickness T1. It is noted that embedding the channel 72 and the transmission path 74 within a single layer of the clothing material 22 does not preclude the clothing material 22 from having multiple layers.

FIG. 5B is a cross-sectional perspective view of a clothing material 22 of the exoskeleton harness 10 of FIGS. 1-3 illustrating a transmission path 74 embedded within two layers 82, 84 of the clothing material 22. In particular, the clothing material 22 includes a first layer 82 and a second layer 84 with the channel 72 formed between the first layer 82 and the second layer 84. Accordingly, the clothing material 22 has a thickness T2 (made up of the first layer 82 and the second layer 84). In certain embodiments, the second layer 84 is merely a strip of material that is smaller (e.g., has less surface area) than the first layer 82. For example, in certain embodiments, the first layer 82 may be the pants 20 and the second layer 84 may be a strip attached to an interior surface of the first layer 82. In certain embodiments, the second layer 84 may be positioned at an interior of the first layer 82, such as for better aesthetics and a more seamless exterior. In certain embodiments, the second layer 84 may be positioned at an exterior of the first layer 82, such as for increased comfort (as the user would not be able to feel the second layer 84).

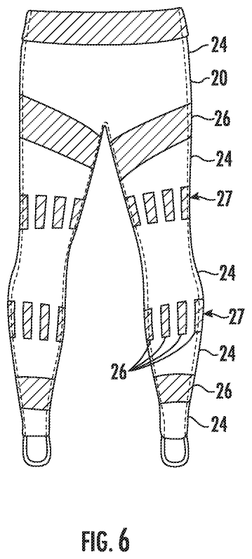

FIG. 6 is a front view of the exoskeleton harness of FIGS. 1-3 illustrating the plurality of patches 26 which provide padding between the user 12 and an exoskeleton 14 (see FIG. 1). The pants 20 of the exoskeleton harness 10 have clothing material 22 that includes a fabric 24 and a plurality of patches 26 (i.e., patch region, padding, padded region, etc.). The fabric 24 is breathable and elastic (i.e., stretchable) to conform to the body of a user 12. For example, in certain embodiments, the fabric 24 is made of spandex, such as Under Armour HeatGear (e.g., Under Armour HeatGear compression leggings).

In certain embodiments, the patches 26 are also elastic to conform to the body of a user 12 and provide greater friction therebetween. For example, in certain embodiments, the patches 26 include neoprene (e.g., closed-cell foamed neoprene, unfoamed neoprene, etc.) and/or Porex medical sponge, etc. In particular, the Porex medical sponge (e.g., one inch thick, 12 mm to 37 mm thick, etc.) is a preferred material for being comfortable against a user's skin, but firm enough to provide a desired force transmission with the exoskeleton 14 (see FIG. 1) without buckling to become a hotspot (e.g., point of abrasion). The patches 26 may be attached to the fabric 24 by being adhered and/or woven, etc.

The patches 26 may be provided in a pattern in a patch area 27. For example, the patches 26 may be provided in a checkered pattern, striped pattern, wavy striped pattern, dot pattern, etc. Providing the patches 26 in a pattern allows more stretchability, flexibility, and/or breathability, while still providing similar padding and/or comfort.

The patches 26 and/or patch areas 27 are positioned to contact corresponding coupling portions (e.g., straps) of the exoskeleton 14. Each coupling portion (e.g., strap) is that part of the exoskeleton 14 that is attached to and/or exerting force upon (e.g., resistive force against) the user 12. The coupling portions are the points of coupling or attachment between the user 12 and the exoskeleton 14.

The patches 26 each have a greater coefficient of static friction, greater thickness, and/or lower permeability than the fabric 24. For example, in certain embodiments, the fabric has a thickness of less than 25 mm and the patches have a thickness greater than 25 mm. In certain embodiments, the fabric has a thickness of less than 12 mm and the patches have a thickness greater than 12 mm. In certain embodiments, the fabric has a thickness less than 6 mm and the patches have a thickness greater than 6 mm. In certain embodiments, the fabric has a thickness of less than 2.5 mm and the patches have a thickness greater than 2.5 mm. In certain embodiments, the fabric has a thickness between 0.1 mm and 1 mm and the patches have a thickness between 12 mm and 25 mm.

The increased friction provided by the patches 26 ensures that the exoskeleton 14 stays in place and does not slip on the user 12, thereby avoiding abrasion, injury and/or the exoskeleton 14 slipping out of alignment. The patches 26 may be less permeable than the fabric 24, and accordingly the patches 26 do not cover the entire surface area of the pants 20 (or other clothing article) of the exoskeleton harness 10. In this way, the patches 26 may be configured to take up as little surface area as possible. In particular, the patches 26 and/or patch areas 27 may be configured to be placed at only the coupling portions. For example, the patches 26 and/or patch areas 27 may comprise annular rings partially or fully separated from one another by the fabric 24. This increases the breathability of the exoskeleton harness 10. In certain embodiments, the patches 26 cover less than 50% of the surface area of the pants 20 and/or the fabric 24. In certain embodiments, the patches 26 cover less than 25% of the surface area of the pants 20 and/or the fabric 24. In certain embodiments, the patches 26 cover less than 25% of the surface area of the pants 20 and/or the fabric 24. In certain embodiments, the patches 26 cover less than 10% of the surface area of the pants 20 and/or the fabric 24. In certain embodiments, the patches 26 cover less than 5% of the surface area of the pants 20 and/or the fabric 24. In certain embodiments, the patches 26 cover less than 1% of the surface area of the pants 20 and/or the fabric 24.

FIG. 7A is a cross-sectional side view of the clothing material 22 of the exoskeleton harness 10 of FIGS. 1-3 and 6 illustrating a fabric 24 and a single layer patch 26. The thickness T3 of the single layer patch 26 is greater than the thickness T4 of the fabric 24. The increased thickness of the patch 26 spreads out the force of coupling portions (e.g., straps) of the exoskeleton 14 on the user 12 and allows much tighter tightening of the straps of the exoskeleton 14 (and thereby better coupling).

FIG. 7B is a cross-sectional side view of the clothing material of the exoskeleton harness 10 of FIGS. 1-3 and 6 illustrating a fabric 24 and a multilayer patch 26′. The multilayer patch 26′ includes a padding layer 86 and a gripping layer 88. In particular, the padding layer 86 has a greater thickness than the fabric 24 and/or the gripping layer 88. In certain embodiments, in certain embodiments, the fabric has a thickness less than 1 mm, the gripping layer has a thickness less than 6 mm, and the patches have a thickness greater than 1 mm. In certain embodiments, in certain embodiments, the fabric has a thickness between 0.1 and 1 mm, the gripping layer has a thickness between 1 and 6 mm, and the patches have a thickness between 12 and 25 mm.

The gripping layer 88 has a greater coefficient of static friction than the padding layer 86 (may also be referred to herein as pads, etc.). In certain embodiments, the padding layer 86 is made of foam and/or the gripping layer 88 is made of rubber.

FIG. 7C is a top view of the clothing material of the exoskeleton harness of FIGS. 1-3 and 6 illustrating a patch 26″ with a checkered pattern of the gripping layer 88 (may also be referred to as gripping pads). In particular, the padding layer 86 extends across the entirety of the patch 26″ and the gripping layer 88 is positioned over less than the entirety of the patch 26″ and the padding layer 86. Providing a patch 26″ with a checkered pattern for the gripping layer 88 provides similar padding and gripping with less surface area, thereby providing improved breathability. Further, providing the patch 26″ as a checkered pattern allows the patch 26″ to be more stretchable and flexible to accommodate a greater variation of body shapes and sizes (e.g., especially when the gripping layer 88 is not stretchable or flexible). Further, the checkered pattern allows stretching in the horizontal and vertical directions. However, other patterns could be used (e.g., striped pattern). As noted above, the patch area 27 may include a plurality of patches 26 in a checkered pattern.

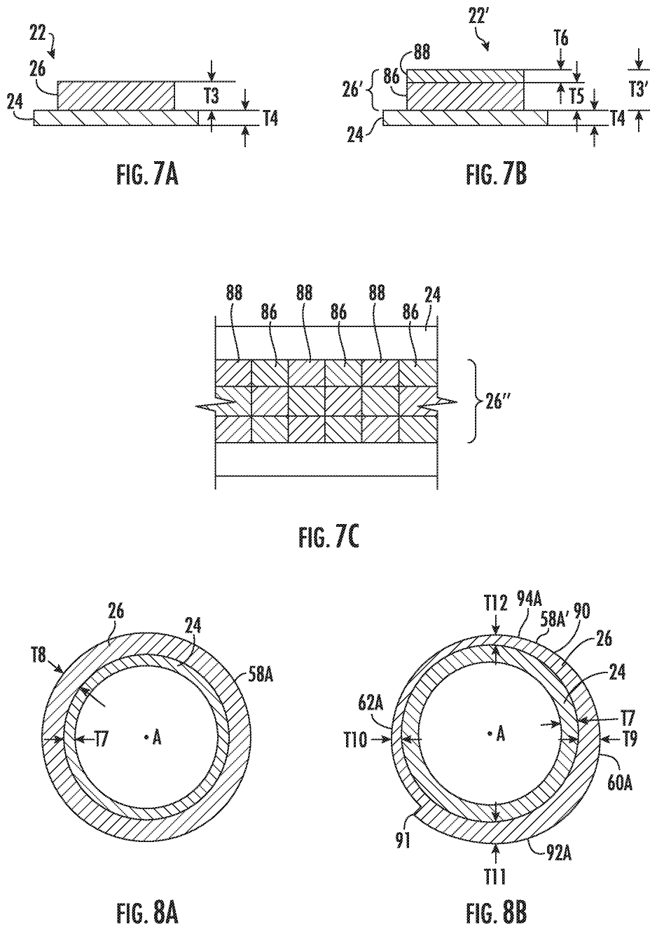

FIG. 8A is a cross-sectional view of an annular patch 26 of the exoskeleton harness 10 of FIGS. 1-3 and 6 illustrating a uniform annular thickness. In particular, the cross-section of clothing material 22 is of a left pant leg 58A. As shown, the thickness T7 of the fabric 24 is uniform about the central axis A. Further, the thickness T8 of the patch 26 is uniform about the central axis A.

FIG. 8B is a cross-sectional view of an annular patch 26 of the exoskeleton harness 10 of FIGS. 1-3 and 6 illustrating a variable annular thickness. In particular, the cross-section of clothing material 22 is of a left pant leg 58A′. The thickness T7 of the fabric 24 is uniform about the central axis A. However, the thickness of the patch 26 is variable about the central axis A. The patch 26 may include a gradient thickness variation 90 (may also be referred to herein as inflections) and/or a stepped thickness variation 91 (may also be referred to herein as steps). Further, the patch 26 may include oscillating gradient thickness variation 90 (to form texture like a wave) and/or oscillating stepped thickness variation 91 (to form texture like a gear) about the central axis A. In other words, the patch 26 may have any number of thickness inflections 91 and/or steps 91 through or about the central axis (e.g., 1 time, 2 times, 3 times, 5 times, 10 times, 20 times, etc.). Note that this may be applied to any patch 26 and/or patch area 27, and is not limited to the annular patch 26 shown in FIG. 8B.

In certain embodiments, the thickness T9 of the patch 26 at the left side seam 60A is greater than the thickness T10 of the patch 26 at the left inseam 62A. Thinner padding at the inseam 62A provides for a more natural gait and stride of the user 12 and a more comfortable user experience when the exoskeleton 14 is mounted to the user 12. In particular, users 12 tend to take a wider, more unnatural stance and stride when additional padding is placed between their legs. In this way, the patch 26 includes at least two thicknesses.

In certain embodiments, the thickness T11 of the patch 26 at the front 92A is greater than the thickness T12 of the patch 26 at the back 94A. A greater amount of force is applied to the front of the leg than the back of the leg of the user 12 when the exoskeleton 14 is mounted to the user 12. Accordingly, less padding is required at the back of the leg. Thinner padding at the back 94A provides a more comfortable user experience when the exoskeleton 14 is mounted to the user 12 (e.g., more comfortable to sit).

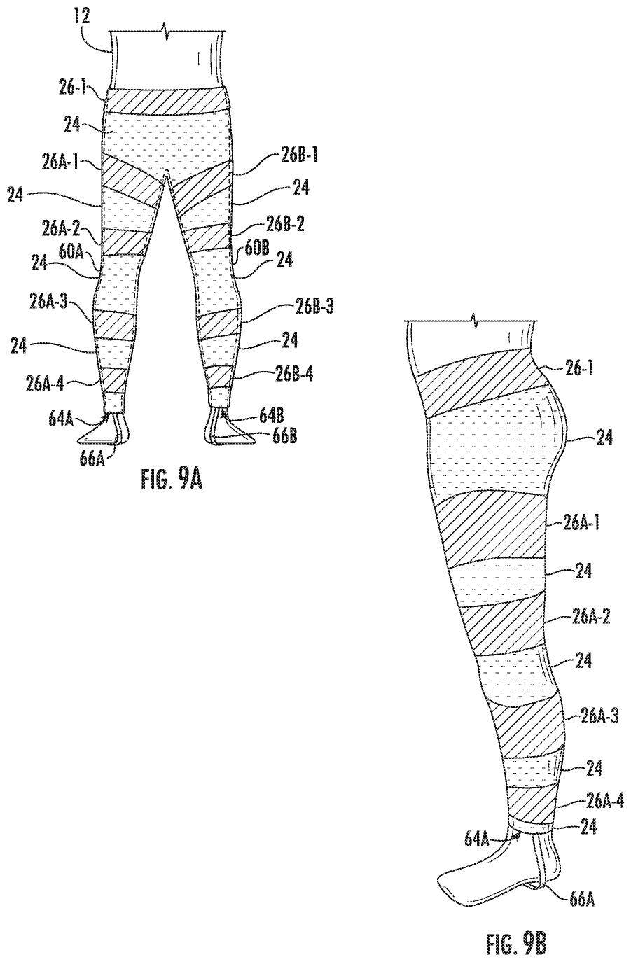

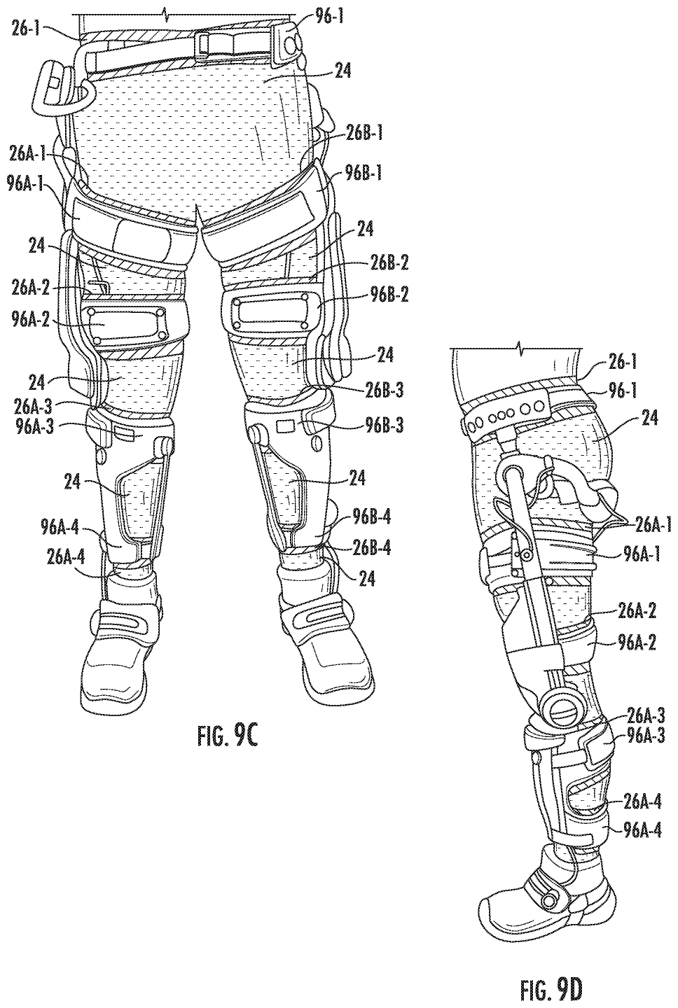

FIGS. 9A-9D are views illustrating pants 20 of the exoskeleton harness 10 of FIGS. 1-3 and 6, as well as the exoskeleton 14 mounted to the exoskeleton harness 10.

FIGS. 9A and 9B are views of the pants 20 of the exoskeleton harness 10 of FIGS. 1-3 and 6. In this embodiment, the pants 20 include a plurality of patches 26, where each patch 26 is an annular band. The annular band is used to accommodate straps that wrap around the legs of the user, however, other patterns could be used. Further, in certain embodiments, the patches 26 may be connected to one another, such as by a longitudinal section at the side seams 60A, 60B.

The patches 26 include a waistband patch 26-1, a first left upper thigh patch 26A-1, a second left upper thigh patch 26A-2, a first left lower thigh patch 26A-3, a second left lower thigh patch 26A-4, a first right upper thigh patch 26B-1, a second right upper thigh patch 26B-2, a first right lower thigh patch 26B-3, and a second right lower thigh patch 26B-4. The first left upper thigh patch 26A-1 and the first right upper thigh patch 26B-1 are positioned proximate the waistband patch 26-1. The second left lower thigh patch 26A-4 and the second right lower thigh patch 26B-4 are positioned furthest from the waistband patch 26-1 (proximate the left leg opening 64A and the right leg opening 64B, respectively). As noted above, the left stirrup 66A and the right stirrup 66B receive at least a portion of a left foot and a right foot of a user 12, respectively. The left stirrup 66A and right stirrup 66B prevent the pants 20 from shifting upward on a user 12 to ensure that the patches 26 are properly positioned relative to the user 12.

FIGS. 9C and 9D are views of the pants 20 of the exoskeleton harness 10 of FIGS. 9A-9B with an exoskeleton 14 mounted to the pants 20 of the exoskeleton harness 10.

The exoskeleton 14 includes a plurality of coupling points 96 (e.g., straps). As shown, the straps 96 generally align and contact the patches 26 of the pants 20 of the exoskeleton harness 10. In particular, the exoskeleton 14 includes a waistband strap 96-1, a first left upper thigh strap 96A-1, a second left upper thigh strap 96A-2, a first left lower thigh strap 96A-3, a second left lower thigh strap 96A-4, a first right upper thigh strap 96B-1, a second right upper thigh strap 96B-2, a first right lower thigh strap 96B-3, and a second right lower thigh strap 96B-4. The first left upper thigh strap 96A-1 and the first right upper thigh strap 96B-1 are positioned proximate the waistband strap 96-1. The second left lower thigh strap 96A-4 and the second right lower thigh strap 96B-4 are positioned furthest from the waistband strap 96-1.

The patches 26 of the pants 20 of the exoskeleton harness 10 align with the straps 96 of the exoskeleton 14. In particular, the straps 96 of the exoskeleton 14 wrap around the patches 26. The patches 26 may have a greater surface area than the straps 96 to compensate for potential variation in alignment. For example, the patches 26 may have a thicker band than the straps 96. The increased friction of the patches 26 keeps the straps 96 in position on the patches 26, and the elasticity of the pants 20 increases friction and coupling between the pants 20 and the user 12. Accordingly, there is better coupling and power transfer between the user 12 and the exoskeleton 14, providing a more comfortable experience with better performance. The high friction material of the patches 26 reduces or eliminates movement of the straps 96 of the exoskeleton 14 when fastened to the patches 26.

Those skilled in the art will recognize improvements and modifications to the preferred embodiments of the disclosure. All such improvements and modifications are considered within the scope of the concepts disclosed herein and the claims that follow.

What is claimed is:

1. An exoskeleton harness, comprising:
a clothing material defining an interior cavity, the clothing material configured to receive at least a portion of a user within the interior cavity, the clothing material including a fabric and at least one patch, only a portion of an exterior surface of the clothing material comprising the at least one patch, the at least one patch having a greater coefficient of static friction and greater thickness than the fabric;
wherein the exoskeleton harness is configured such that the at least one patch is positioned to align with and contact at least one coupling portion of an exoskeleton.

2. The exoskeleton harness of claim 1, wherein the clothing material is configured to elastically receive and conform to the at least a portion of the user within the interior cavity.

3. The exoskeleton harness of claim 1, wherein the at least one patch comprises a plurality of patches separated from one another by the fabric.

4. The exoskeleton harness of claim 3, wherein the plurality of patches form a patch area configured such that the patch area is positioned to align with and contact the at least one coupling portion of the exoskeleton.

5. The exoskeleton harness of claim 3, wherein the at least one patch comprises a plurality of annular rings.

6. The exoskeleton harness of claim 1, wherein the at least one patch comprises a first layer and a second layer, the first layer having a lesser thickness and a greater coefficient of static friction than the second layer.

7. The exoskeleton harness of claim 1, wherein the at least one patch comprises at least two thicknesses.

8. The exoskeleton harness of claim 1,
wherein the exoskeleton harness comprises pants;
wherein the at least one patch is thicker at a side seam than an inseam; and
wherein the at least one patch is thicker at a front side of the pants than a back side of the pants.

9. The exoskeleton harness of claim 1, further comprising at least one internal electronic port positioned at an interior of the clothing material within the interior cavity, the at least one internal electronic port configured to electronically communicate with at least one first electronic device separate from the exoskeleton harness.

10. The exoskeleton harness of claim 9, wherein the at least one internal electronic port comprises a plurality of internal electronic ports and the at least one first electronic device comprises a plurality of biosensors, each of the plurality of internal electronic ports configured to electronically communicate with one of the plurality of biosensors.

11. The exoskeleton harness of claim 9,
further comprising an external electronic port positioned at an exterior of the clothing material and configured to electronically communicate with the at least one first electronic device separate from the exoskeleton harness; and
at least one transmission path embedded within the clothing material communicatively connecting the external port to the at least one internal port.

12. An exoskeleton harness, comprising:
a clothing material defining an interior cavity, the clothing material configured to receive at least a portion of a user within the interior cavity, the clothing material having a thickness extending between an exterior and an interior of the clothing material;
an external electronic port positioned at the exterior of the clothing material and configured to electronically communicate with a first electronic device separate from the exoskeleton harness;
at least one internal electronic port positioned at the interior of the clothing material within the interior cavity, the at least one internal electronic port configured to electronically communicate with at least one second electronic device separate from the exoskeleton harness; and
at least one transmission path embedded within the clothing material and communicatively connecting the external port to the at least one internal port.

13. The exoskeleton harness of claim 12, wherein the clothing material is configured to elastically receive and conform to the at least a portion of the user within the interior cavity.

14. The exoskeleton harness of claim 12,
wherein the clothing material further comprises a fabric and at least one patch, only a portion of an exterior surface of the clothing material comprising the at least one patch, the at least one patch having a greater coefficient of static friction and greater thickness than the fabric; and
wherein the exoskeleton harness is configured such that the at least one patch is positioned to align with and contact at least one coupling portion of an exoskeleton.

15. The exoskeleton harness of claim 12, wherein the at least one transmission path is embedded within a single layer of the clothing material.

16. The exoskeleton harness of claim 12,
wherein the clothing material includes a first layer and a second layer attached to the first layer, the first layer and the second layer defining a wiring channel therebetween; and
wherein the at least one transmission path is embedded within the wiring channel between two layers of the clothing material.

17. The exoskeleton harness of claim 12, wherein the at least one internal electronic port comprises a plurality of internal electronic ports and the at least one second electronic device comprises a plurality of second electronic devices, each of the plurality of internal electronic ports configured to electronically communicate with one of the plurality of second electronic devices.

18. The exoskeleton harness of claim 17, wherein each internal electronic port is connected to the external electronic port by the at least one transmission path.

19. The exoskeleton harness of claim 18,
wherein the external electronic port is configured to electronically communicate at least one of wired or wirelessly with the first electronic device; and
wherein the at least one transmission path comprises at least one of an electrical wire or a fiber optic cable.

20. The exoskeleton harness of claim 12, wherein the second electronic device comprises a skin-mounted biosensor.

21. The exoskeleton harness of claim 20, wherein the skin-mounted biosensor comprises an electromyography biosensor.

22. An exoskeleton harness, comprising:
a clothing material defining an interior cavity, the clothing material configured to receive at least a portion of a user within the interior cavity, the clothing material including a fabric and at least one patch, only a portion of an exterior surface of the clothing material comprising the at least one patch, the at least one patch having a greater coefficient of static friction and greater thickness than the fabric, the clothing material having a thickness extending between an exterior and an interior of the clothing material;
an external electronic port positioned at the exterior of the clothing material and configured to electronically communicate with an exoskeleton;
at least one internal electronic port positioned at the interior of the clothing material within the interior cavity, the at least one internal electronic port configured to electronically communicate with one of a plurality of skin-mounted biosensors; and
at least one transmission path embedded within the clothing material and communicatively connecting the external port to the at least one internal port;
wherein the exoskeleton harness is configured such that the at least one patch is positioned to align with and contact at least one coupling portion of the exoskeleton.
